(19) United States
(12) Patent Application Publication
(10) Pub. No.: US 2010/0311678 A1
(43) Pub. Date: Dec. 9, 2010

(54) METHODS AND COMPOSITIONS FOR TREATING CANCER AND MODULATING SIGNAL TRANSDUCTION AND METABOLISM PATHWAYS

(76) Inventors: Bruce P. Bean, Waban, MA (US); Alexander Binshtok, Brookline, MA (US); Clifford J. Woolf, Newton, MA (US)

Correspondence Address: CLARK & ELBING LLP, 101 FEDERAL STREET, BOSTON, MA, 02110, US

(21) Appl. No.: 12/681,509

(22) PCT Filed: Oct. 3, 2008
(86) PCT No.: PCT/US08/11454

§ 371 Date: Aug. 23, 2010

Related U.S. Application Data
(60) Provisional application No. 60/997,715, filed on Oct. 4, 2007.
(60) Provisional application No. 61/051,180, filed on May 7, 2008.

(51) Int. Cl. A61K 31/704; A61K 31/166; A61P 35/00

(52) U.S. Cl. 514/34; 514/619

(57) ABSTRACT

This invention features methods and compositions for treating cancer and modulating signal transduction and metabolism pathways. For example, the methods and compositions of the invention can be used to kill or inhibit the growth or spread of cancer cells. The invention also features a method of identifying a compound that modulates a signal transduction or metabolic pathway.

### FIELD OF THE INVENTION

This invention features methods and compositions for treating cancer and modulating signal transduction and metabolism pathways. For example, the methods and compositions of the invention can be used to kill or inhibit the growth or spread of cancer cells. The invention also features a method of identifying a compound that modulates a signal transduction or metabolic pathway.

### BACKGROUND OF THE INVENTION

A limitation of many pharmaceutical and biological therapies is the non-selective nature by which therapeutic compounds, for example, antiproliferative agents for the treatment of cancer, are administered to a patient. The vast majority of such externally-applied compounds are hydrophobic and can pass through membranes. Accordingly, these compounds enter all cells and thus have no selectivity for affecting only, for example, cancer cells.

It is desirable to develop methods for delivering therapeutic compounds that preferentially affect a target cell (e.g., cancer cells, tissues, or organs) but are unable to exert a biological effect on bystander cells (e.g., non-cancer cells).

### SUMMARY OF THE INVENTION

This invention features methods and compositions for the treatment of cancer in a patient (e.g., a human). For example, the methods and compositions of the invention can be used to kill or inhibit the growth or spread of cancer cells in a patient suffering from cancer. This is accomplished by administering to a patient a first compound that activates channel-forming receptors (e.g., large-pore cation channels, such as TRPV1), causing these receptors to open and allow an amphoteric or permanently positively charged antiproliferative agent into the intracellular space. Charged forms of antiproliferative agents show reduced permeability across membranes to enter the cytoplasm or nucleus where they act to kill the cells. Accordingly, the methods and compositions of the invention provide increased intracellular uptake of the antiproliferative agent into a target cell (e.g., a cancer cell), thereby potentiating antiproliferative agents that lack or substantially lack extracellular anti-cancer activity. Many cancer cells express large-pore cation channels such as TRP channels and P2X-receptor channels through which such agents can enter cells (e.g., Sanchez et al., “Expression of the transient receptor potential vanilloid 1 (TRPV1) in LNCaP and PC-3 prostate cancer cells and in human prostate tissue,” Eur. J. Pharmacol. 515(1-3):20-27 (2005); Zhang and Barritt, “Evidence that TRPM8 is an androgen-dependent Ca²⁺ channel required for the survival of prostate cancer cells,” Cancer Res. 64(22):8365-8373 (2004); Raffaghello et al., “The P2X7 receptor sustains the growth of human neuroblastoma cells through a substance P-dependent mechanism,” Cancer Res. 66:907-914 (2006); and Shabbir et al., “Purinergic receptor-mediated effects of ATP in high-grade bladder cancer,” BJU Int. 101:106-112 (2008)). Normal non-cancer cells that do not express or express few channel-forming receptors are not susceptible to increased intracellular uptake of antiproliferative agents, thus reducing background or bystander toxicity to these agents.

The invention also features methods and compositions to modulate intracellular signal transduction and metabolism pathways to treat a condition where the selective activation or disruption of a cellular signal transduction or metabolism pathway is beneficial. This is accomplished by administering to a patient a first compound that activates channel-forming receptors (e.g., large-pore cation channels, such as TRPV1), causing these receptors to open and allow a second amphoteric or permanently positively charged compound into the intracellular space. As the second compound is not active until it reaches the intracellular space, other cells exposed to this compound that do not express or express few channel-forming receptors are not susceptible to intracellular signal transduction or metabolism modulation.

In a first aspect, the invention features a method for treating cancer in a patient by administering to a patient a first compound that activates a channel-forming receptor that is present on a target cell (e.g., a cancer cell) and an antiproliferative agent that is capable of entering the target cell when the receptor is activated. In one embodiment, the compositions of the invention are administered to a mammal (e.g., a human) to treat cancer. Cancers that can be treated according to the methods of the invention include but are not limited to esophageal cancer, prostate cancer, skin cancer, brain cancer, colon cancer, lung cancer, breast cancer, ovarian cancer, rectal cancer, bladder cancer, renal cancer, melanoma, pancreatic cancer, thyroid cancer, non-Hodgkin's lymphoma, leukemia, and endometrial cancer. In another embodiment, the antiproliferative agent does not substantially inhibit the cancer when applied extracellularly in the absence of said first compound. In a further embodiment, the antiproliferative agent can have a positive charge. An antiproliferative agent can be chemically modified to confer a positive charge.

In a second aspect, the invention provides a method for modulating intracellular signal transduction and metabolic pathways by administering a first compound that activates (e.g., opens) a channel-forming receptor that is present on a target cell and a second compound that inhibits or activates an intracellular signal transduction or metabolic pathway. The second compound is capable of entering the target cell through the receptor when the receptor is activated and does not substantially inhibit the intracellular signal transduction or metabolic pathway when applied extracellularly in the absence of the first compound. In one embodiment, the method provides for the modulation (i.e., inhibition or activation) of enzymatic activity. Particularly attractive target enzymes of a second compound include intracellular protein kinases and phosphatases. Examples of intracellular protein kinases include but are not limited to protein kinase C (PKC), protein kinase A (PKA), MAP kinase, MAP kinase kinase (MAP2K), MAP kinase kinase kinase (MAP3K), extracellular signal-regulated kinase (ERK), c-jun N-terminal kinase (JNK), Src kinase, STAT, WNT, MYC, RAS, cyclin-dependent kinases, AKT pathway kinases, p53 pathway kinases, EGF pathway kinases and p38 kinase. Intracellular phosphatases include but are not limited to tyrosine-specific phosphatases, serine-threonine specific phosphatases, dual specificity phosphatases, histidine phosphatases, and lipid phosphatases. Other enzymes involved in cell growth, differentiation, apoptosis, and division can also be modulated by contacting a patient with a second compound that activates or inhibits an enzyme. In another embodiment, the second compound can have a positive charge. The second compound can be chemically modified to confer a positive charge.

In an embodiment of the first and second aspects of the invention, it may be desirable to administer the first compound in order to ensure that the receptors (e.g., the TRPV1, TRPV6, TRPM1, TRPC1, TRPC6, TRPM4, TRPM5, TRPP8, TRPA1, P2X(2/3), and TRPM8 receptors) are activated, thus allowing for entry of the second compound. In other embodiments, because the receptors (e.g., the TRPV1, TRPV6, TRPM1, TRPC1, TRPC6, TRPM4, TRPM5, TRPP8, TRPA1, P2X(2/3), and TRPM8 receptors) are already activated, the first compound is not administered. Consequently, the second compound enters only cells having receptors that are endogenously activated.

In an embodiment of the first and second aspects of the invention, two or more compounds that activate TRPV1, TRPV6, TRPM1, TRPC1, TRPC6, TRPM4, TRPM5, TRPP8, TRPA1, P2X(2/3), and TRPM8 receptors can be employed, as can two or more compounds that modulate signal transduction or metabolism pathways. Desirably, the first compound(s) and the second compound(s) are administered to the patient within 4 hours, 2 hours, 1 hour, 30 minutes, or 15 minutes of each other, or are administered substantially simultaneously. Importantly, either compound can be administered first. Thus, in one embodiment, one or more compounds that activate TRPV1, TRPV6, TRPM1, TRPC1, TRPC6, TRPM4, TRPM5, TRPP8, TRPA1, P2X(2/3), and TRPM8 receptors are administered first, while in another embodiment, one or more compounds that inhibit one or more signal transduction or metabolism pathways when present intracellularly but not extracellularly are administered first. The compounds can be co-formulated into a single composition or can be formulated separately. Each of the compounds can be administered, for example, by oral, parenteral, intravenous, intramuscular, rectal, cutaneous, subcutaneous, topical, transdermal, sublingual, nasal, vaginal, intrathecal, epidural, or ocular administration, or by injection, inhalation, or direct contact with the nasal or oral mucosa.

In a third aspect, the invention features compositions for the treatment of cancer in a patient. These compositions include a first compound that activates a channel-forming receptor and an antiproliferative agent that is capable of entering the target cell through the receptor when the receptor is activated.

In an embodiment of the first or third aspects of the invention, the antiproliferative agent is an alkylating agent, platinum agent, antimetabolite, topoisomerase inhibitor, antitumor antibiotic, antimitotic agent, aromatase inhibitor, thymidylate synthase inhibitor, DNA antagonist, farnesyltransferase inhibitor, histone acetyltransferase inhibitor, metalloproteinase inhibitor, ribonucleoside reductase inhibitor, photodynamic agent, or tyrosine kinase inhibitor. Alkylating agents include cyclophosphamide, busulfan, mannosulfan, treosulfan, hexamethylmelamine, altretamine, thiotepa, mechlorethamine, estramustine, uramustine, melphalan, chlorambucil, chlormethine, ifosfamide, bendamustine, trosfosfamide, carmustine, fotemustine, lomustine, nimustine, prednimustine, ranimustine, semustine, streptozocin, dacabazine, temozolomide, procarbazine, dacarbazine, carboquone, thioTEPA, triaziquone, and triethylenemelamine. Platinum agents include cisplatin, carboplatin, nedaplatin, oxaliplatin, satraplatin, and triplatin tetranitrate. Antimetabolites include aminopterin, methotrexate, pemetrexed, raltitrexed, cladribine, clofarabine, fludarabine, mercaptopurine, pentostatin, thioguanine, cytarabine, decitabine, fluorouracil, capecitabine, floxuridine, gemcitabine, enocitabine, and sapacitabine. Topoisomerase inhibitors include camptothecin, topotecan, irinotecan, rubitecan, belotecan, etoposide, amsacrine, and teniposide. Antitumor antibiotics include aclarubicin, daunorubicin, doxorubicin, epirubicin, idarubicin, amrubicin, pirarubicin, valrubicin, zorubicin, mitoxantrone, pixantrone, actinomycin, bleomycin, mitomycin, plicamycin, and hydroxyurea. Antimitotic agents include docetaxel, larotaxel, ortataxel, paclitaxel, tesetaxel, vinblastine, vincristine, vinflunine, vindesine, vinorelbine, ixabepilone, procainamide, metoclopramide, and declopramide. Aromatase inhibitors include aminoglutethimide, anastrozole, letrozole, vorozole, exemestane, 4-androstene-3,6,17-trione, 1,4,6-androstatrien-3,17-dione, formestane, testolactone, and fadrozole. Photodynamic agents include aminolevulinic acid, methyl aminolevulinate, efaproxiral, porfimer sodium, talaporin, temoporfin, or verteporfin. Farnesyltransferase inhibitors include tipifarnib and lonafarnib. Tyrosine kinase inhibitors include axitinib, bosutinib, cediranib, dasatinib, erlotinib, gefitinib, imatinib, lapatinib, lestaurtinib, nilotinib, semaxanib, sorafenib, sunitinib, and vandetanib. Other antiproliferative agents suitable for use according to the methods of the invention are known to those skilled in the art.

In a fourth aspect, the invention features compositions for the modulation of an intracellular signal transduction or metabolism pathway. These compositions include a first compound that activates (i.e., opens) a channel-forming receptor on a target cell and a second compound that inhibits or activates an intracellular signal transduction or metabolic pathway. The second compound is capable of entering the target cell through the receptor when the receptor is activated and does not substantially inhibit the intracellular signal transduction or metabolic pathway when applied extracellularly in the absence of the first compound.

In an embodiment of any aspect of the invention, activators of TRPV1 receptors include but are not limited to capsaicin, lidocaine, eugenol, arvanil (N-arachidonoylvanillamine), anandamide, 2-aminoethoxydiphenyl borate (2APB), AM404, resiniferatoxin, phorbol 12-phenylacetate 13-acetate 20-homovanillate (PPAHV), olvanil (NE 19550), OLDA (N-oleoyldopamine), N-arachidonyldopamine (NADA), 6′-iodoresiniferatoxin (6′-IRTX), C18 N-acylethanolamines, lipoxygenase derivatives such as 12-hydroperoxyeicosatetraenoic acid, inhibitor cysteine knot (ICK) peptides (vanillotoxins), piperine, MSK195 (N-[2-(3,4-dimethylbenzyl)-3-(pivaloyloxy)propyl]-2-[4-(2-aminoethoxy)-3-methoxyphenyl]acetamide), JYL79 (N-[2-(3,4-dimethylbenzyl)-3-(pivaloyloxy)propyl]-N′-(4-hydroxy-3-methoxybenzyl)thiourea), SU200 (N-(4-tert-butylbenzyl)-N′-(4-hydroxy-3-methoxybenzyl)thiourea), transacin, ALGRX 4975, NGX-1998, and TQ-1018. Activators of TRPA1 receptors include but are not limited to cinnamaldehyde, allyl-isothiocynanate, diallyl disulfide, icilin, cinnamon oil, wintergreen oil, clove oil, acrolein, and mustard oil. Activators of P2X receptors include but are not limited to ATP, 2-methylthio-ATP, 2′ and 3′-O-(4-benzoylbenzoyl)-ATP, and ATP5′-O-(3-thiotriphosphate). Activators of TRPM8 receptors include but are not limited to menthol, icilin, eucalyptol, linalool, geraniol, and hydroxycitronellal. Activators of other large-pore cation channel receptors, such as TRPV6, TRPM1, TRPC1, TRPC6, TRPM4, TRPM5, and TRPP8 exist and are known to those skilled in the art.

In an embodiment of the third and fourth aspects of the invention, the compositions are formulated for oral, parenteral (e.g., intravenous, intramuscular), rectal, cutaneous, subcutaneous, topical, transdermal, sublingual, nasal, vaginal, intrathecal, epidural, or ocular administration, or administration by injection, inhalation, or direct contact with the nasal or oral mucosa.

In a final aspect, the invention features a method for identifying a compound that modulates intracellular signal transduction or metabolic pathway. This method includes the steps of: (a) contacting the external face of TRPV1, TRPV6, TRPM1, TRPC1, TRPC6, TRPM4, TRPM5, TRPP8, TRPA1, P2X(2/3), or TRPM8 expressing target cells with: (i) a first compound that activates TRPV1 TRPA1, TRPM8 or P2X(2/3) receptors; and (ii) a second compound that modulates an intracellular signal transduction or metabolism pathway when present intracellularly but not extracellularly, and (b) determining whether the second compound induces modulation of intracellular signal transduction or metabolism pathways in the target cells.

It is understood that other receptors may exist that would permit the entry of compounds that would otherwise be incapable of entering. Co-administration of compounds that activate one or more of these receptors in combination with one or more compounds that modulate intracellular signal transduction or metabolic pathways when applied to the internal face of the channels but does not substantially modulate these pathways when applied to the external face of the channels is also an aspect of the invention.

By “phosphatase inhibitor” or “kinase inhibitor” is meant an agent that binds a phosphatase or kinase and inhibits (e.g. by at least 10%, 20%, or 30% or more) the biological activity of that enzyme.

By “inhibits cell proliferation” is meant measurably slows, stops, or reverses the growth rate of cells in vitro or in vivo. Desirably, a slowing of the growth rate is by at least 20%, 30%, 50%, 60%, 70%, 80%, or 90%, as determined using a suitable assay for determination of cell growth rates. Typically, a reversal of growth rate is accomplished by initiating or accelerating necrotic or apoptotic mechanisms of cell death in the neoplastic cells or by inhibiting cell division (e.g., using a mitotic inhibitor), DNA, RNA or protein synthesis.

By “cancer” is meant abnormal cellular proliferation that results in a disease (malignant or benign). Specific examples include but are not limited to tumors of the breast, colon, rectum, lung, oropharynx, hypopharynx, esophagus, stomach, pancreas, liver, bilecyst, bile duct, small intestine; urinary system including kidney, bladder and epithelium of urinary tract; female genital system including uterine neck, uterus, ovary, chorioma and gestational trophoblastic diseases; male genital system including prostate, seminal vesicle and testis; endocrine glands including thyroid gland, adrenal gland and pituitary body; skin cancer including angioma, melanoma, sarcoma originated from bone or soft tissue, and Kaposi's sarcoma; tumors of brain, nervus, eye and meninges, including astrocytoma, neuroastrocytoma, spongioblastoma, retinoblastoma, neuroma, neuroblastoma, neurinoma and neuroblastoma; solid tumors developed from malignant diseases of hemopoietic system, including chloroleukemia, plasmacytoma and dermal T lymphoma/leukemia; lymphoma including Hodgkin's lymphoma and non-Hodgkin's lymphoma.

By “treating cancer,” “preventing cancer,” or “inhibiting cancer” is meant causing a reduction in the size of a tumor or the number of cancer cells, slowing, preventing, or inhibiting an increase in the size of a tumor or cancer cell proliferation, increasing the disease-free survival time between the disappearance of a tumor or other cancer and its reappearance, preventing or reducing the likelihood of an initial or subsequent occurrence of a tumor or other cancer, or reducing an adverse symptom associated with a tumor or other cancer. In a desired embodiment, the percent of tumor or cancerous cells surviving the treatment is at least 20, 40, 60, 80, or 100% lower than the initial number of tumor or cancerous cells, as measured using any standard assay, such as those described herein. Desirably, the decrease in the number of tumor or cancerous cells induced by administration of a compound of the invention is at least 2, 5, 10, 20, or 50-fold greater than the decrease in the number of non-tumor or non-cancerous cells. Desirably, the methods of the present invention result in a decrease of 20, 40, 60, 80, or 100% in the size of a tumor or number of cancerous cells as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after no less than 5, 10, 15, or 20 years.

By “patient” is meant any animal. In one embodiment, the patient is a human. Other animals that can be treated using the methods, compositions, and kits of the invention include but are not limited to non-human primates (e.g., monkeys, gorillas, chimpanzees), domesticated animals (e.g., horses, pigs, goats, rabbits, sheep, cattle, llamas), and companion animals (e.g., guinea pigs, rats, mice, lizards, snakes, dogs, cats, fish, hamsters, and birds).

By “intracellular signal transduction pathway” or “metabolism pathway” is meant any sequence of molecular binding or enzymatic events that propagate a signal within a cell. These signaling events include but are not limited to pathways involving regulation of metabolism, cell growth, movement, apoptosis, proliferation and division, and specialized cellular functions. Kinases and phosphatases are particularly import mediators of signal transduction and metabolism pathways and therefore represent particularly attractive targets for the modulation of such pathways. Proto-oncogenes and oncogenes such as WNT, MYC, RAS, the cyclin-dependent kinases, AKT pathway kinases, p53 pathway kinases, and EGF pathway kinases are also suitable targets.

By “low molecular weight” is meant less than 500 Daltons.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include but are not limited to acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include but are not limited to sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

By “charged moiety” is meant a moiety which gains a proton at physiological pH thereby becoming positively charged (e.g., ammonium, guanidinium, or amidinium) or a moiety that includes a net formal positive charge without protonation (e.g., quaternary ammonium). The charged moiety may be either permanently charged or transiently charged.

Other features and advantages of the invention will be apparent from the following detailed description, and from the claims.

### BRIEF DESCRIPTION OF THE DRAWINGS

FIG. 1 is a series of micrographs showing intracellular delivery of doxorubicin a fluorescent antiproliferative agent into adult rat dissociated dorsal root ganglion (DRG) neurons. The DRG neurons were treated with vehicle control (FIG. 1A), 2 mM doxorubicin (Adriamycin®) for 20 minutes (FIG. 1B), or 2 mM doxorubicin/100 mM L-menthol for 10 minutes (FIG. 1C). The doxorubicin only entered into cells in the presence of menthol which is a TRPM8 agonist. The size and number of the fluorescently labeled neurons are similar to those of TRPM8 expressing neurons.

FIG. 2 is a series of micrographs showing intracellular delivery of doxorubicin into adult rat dissociated dorsal root ganglion (DRG) neurons. The DRG neurons were treated with vehicle control (FIG. 2A), 2 mM doxorubicin (Adriamycin®) for 20 minutes (FIG. 2B), or 2 mM doxorubicin (Adriamycin®)/1 mM capsaicin for 5 minutes (FIG. 2C). The doxorubicin only entered into cells in the presence of capsaicin which is a TRPV1 agonist. The size and number of the fluorescently labeled neurons are similar to those of TRPV1 expressing neurons.

### DETAILED DESCRIPTION OF THE INVENTION

We have discovered a means for delivering compounds into cells that express channel-forming receptors (e.g., large-pore cation channels, such as TRPV1). By providing a way for these compounds to preferentially enter channel-forming receptor-expressing cell subsets, the invention permits the use, both in screening and in therapeutic applicaions, of entire classes of compounds that are biologically active intracellularly but are poorly membrane-permeant or entirely membrane impermeant. Facilitating the preferential entry of such compounds (e.g., antiproliferative agents) to cells that express channel-forming receptors (e.g., cancer cells) can allow for improved compound pharmokokinetics, reduced compound side effects or bystander cell damage, and increased therapeutic benefits to the treated patient. In one embodiment of the invention, a compound can be chemically-modified to confer a positive charge that can facilitate passage through a channel-forming receptor, such as large-pore cation channels, and prevent or reduce entry into cells that not expressing such receptors.

One aspect of the invention concerns the treatment of cancer in a patient. Many chemotherapeutic treatments used in the treatment of proliferative disorders are cytotoxic to all cells exposed to critical concentrations of the compound. Accordingly, many chemotherapeutics exhibit profound side effects in treated patients, oftentimes prohibiting their effective use in the treatment of cancer. The preferential introduction of compounds into cancer cells that antagonize or inhibit normal intracellular signaling or metabolism pathways provides a means to overcome the limitations of broadly cytotoxic chemotherapeutic agents. This is accomplished by administering to a patient diagnosed with cancer a first compound that activates a channel-forming receptor (e.g., a large-pore cation channel, such as TRPV1) that allow the preferential entry of an antiproliferative agent into cancer cells. Since some cancer cells express greater levels of large-pore cation channels than non-cancerous “bystander” cells, cancer cells will preferentially take up the antiproliferative agent. The present invention, by use of channel-forming receptor agonists, provides for the introduction of compounds into cancer cells that can exert a beneficial biological activity once inside the treated cell.

### Large-Pore Cation Channel Agonists

### TRPV1 Agonists

TRPV1 agonists that can be employed in the methods and compositions of the invention include but are not limited to any that activate TRPV1 receptors on a target cell (e.g., a cancer cell) and allows for entry of at least one compound. Suitable TRPV1 agonists include but are not limited to capsaicin, lidocaine, eugenol, arvanil (N-arachidonoylvanillamine), anandamide, 2-aminoethoxydiphenyl borate (2APB), AM404, resiniferatoxin, phorbol 12-phenylacetate 13-acetate 20-homovanillate (PPAHV), olvanil (NE 19550), OLDA (N-oleoyldopamine), N-arachidonyldopamine (NADA), 6′-iodoresiniferatoxin (6′-IRTX), C18 N-acylethanolamines, lipoxygenase derivatives such as 12-hydroperoxyeicosatetraenoic acid, inhibitor cysteine knot (ICK) peptides (vanillotoxins), piperine, MSK195 (N-[2-(3,4-dimethylbenzyl)-3-(pivaloyloxy)propyl]-2-[4-(2-aminoethoxy)-3-methoxyphenyl]acetamide), JYL79 (N-[2-(3,4-dimethylbenzyl)-3-(pivaloyloxy)propyl]-N′-(4-hydroxy-3-methoxybenzyl)thiourea), and SU200 (N-(4-tert-butylbenzyl)-N′-(4-hydroxy-3-methoxybenzyl)thiourea).

### TRP1A Agonists

TRP1A agonists that can be employed in the methods and compositions of the invention include any that activates TRP1A receptors on a target cell (e.g., a cancer cell) and allows for entry of at least one compound. Suitable TRP1A agonists include but are not limited to cinnamaldehyde, allyl-isothiocynanate, diallyl disulfide, icilin, cinnamon oil, wintergreen oil, clove oil, acrolein, and mustard oil.

### P2X Agonists

P2X agonists that can be employed in the methods and compositions of the invention include any that activates P2X receptors on a target cell (e.g., a cancer cell) and allows for entry of at least one compound. Suitable P2X agonists include but are not limited to 2-methylthio-ATP, 2′ and 3′-O-(4-benzoylbenzoyl)-ATP, and ATP5′-O-(3-thiotriphosphate).

### TRPM8 Agonists

TRPM8 agonists that can be employed in the methods compositions of the invention include any that activates TRPM8 receptors on a target cell (e.g., a cancer cell) and allows for entry of at least one compound. Suitable TRPM8 agonists include but are not limited to menthol, iciclin, eucalyptol, linalool, geraniol, and hydroxycitronellal.

### Other TRP Agonists

Other TRP large pore cation channel receptor family members may be activated to accommodate the entry of an intracellular signal or metabolism modulating compound into a target cell (e.g., a cancer cell). Other TRP receptors include, but are not limited to TRPV6, TRPM1, TRPC1, TRPC6, TRPM4, TRPM5, and TRPP8. Agonists or activators of these receptors are known to those of skill in the art.

### Antiproliferative Agents

Antiproliferative agents, also known as antineoplastic agents, are compounds that inhibit cell proliferation. Antiproliferative agents that be used in the methods and compositions of the invention include, e.g., alkylating agents (e.g., cyclophosphamide, busulfan, mannosulfan, treosulfan, hexamethylmelamine, altretamine, thiotepa, mechlorethamine, estramustine, uramustine, melphalan, chlorambucil, chlormethine, ifosfamide, bendamustine, trosfosfamide, carmustine, fotemustine, lomustine, nimustine, prednimustine, ranimustine, semustine, streptozocin, dacabazine, temozolomide, procarbazine, dacarbazine, carboquone, thioTEPA, triaziquone, and triethylenemelamine); platinum agents (e.g., cisplatin, carboplatin, nedaplatin, oxaliplatin, satraplatin, and triplatin tetranitrate); antimetabolites (e.g., aminopterin, methotrexate, pemetrexed, raltitrexed, cladribine, clofarabine, fludarabine, mercaptopurine, pentostatin, thioguanine, cytarabine, decitabine, fluorouracil, capecitabine, floxuridine, gemcitabine, enocitabine, and sapacitabine); topoisomerase inhibitors (e.g., camptothecin, topotecan, irinotecan, rubitecan, belotecan, etoposide, amsacrine, and teniposide); antitumor antibiotics (e.g., aclarubicin, daunorubicin, doxorubicin, epirubicin, idarubicin, amrubicin, pirarubicin, valrubicin, zorubicin, mitoxantrone, pixantrone, actinomycin, bleomycin, mitomycin, plicamycin, and hydroxyurea); antimitotic agents (e.g., docetaxel, larotaxel, ortataxel, paclitaxel, tesetaxel, vinblastine, vincristine, vinflunine, vindesine, vinorelbine, ixabepilone, procainamide, metoclopramide, and declopramide); aromatase inhibitors (e.g., aminoglutethimide, anastrozole, letrozole, vorozole, exemestane, 4-androstene-3,6,17-trione, 1,4,6-androstatrien-3,17-dione, formestane, testolactone, and fadrozole); thymidylate synthase inhibitors; DNA antagonists; farnesyltransferase inhibitors; histone acetyltransferase inhibitors; metalloproteinase inhibitors; ribonucleoside reductase inhibitors; photodynamic agents (e.g., aminolevulinic acid, methyl aminolevulinate, efaproxiral, porfimer sodium, talaporin, temoporfin, and or verteporfin); and tyrosine kinase inhibitors (e.g., axitinib, bosutinib, cediranib, dasatinib, erlotinib, gefitinib, imatinib, lapatinib, lestaurtinib, nilotinib, semaxanib, sorafenib, sunitinib, and vandetanib).

### Intracellular Signaling and Metabolic Pathway Modulators

The methods and compositions of the invention provide intracellular signaling and metabolic pathway modulators to treat conditions or diseases characterized by, e.g., the deficiency or excess of cellular molecules.

### Protein Kinases and Phosphatases

Protein kinases are enzymes that phosphorylate themselves or other molecules. The act of phosphorylation oftens elicits a chemical or structural change in the target molecule that allows further binding or enzymatic reactions to occur, thus propogating a “signal” through a cell. At the end of a particular signaling pathway, some terminal effector molecule(s) (e.g., transcriptional repressor or promoter, apoptotic factor) exerts its particular activity in the cell, resulting in changes to cellular growth, differentiation, or survival characteristics. Alternatively, phosphatases are enzymes that remove phosphate groups from phosphorylated molecules, and therefore are also involved in the modulation of signal transduction and metabolism pathways within a cell. Kinases and phosphatases are therefore particularly attractive targets for methods to modulate intracellular signal transduction and metabolism pathways. Activators and inhibitors of the protein kinases and phosphatases exist and are known to those with skill in the art.

### Intracellular Enzymes, Proteins, and Metabolic Regulators

There exist many intracellular enzymes, proteins, and metabolic regulators in a target cell that are attractive targets for the modulation of an intracellular signal transduction or metabolism pathway. For example, activation of the endogenous caspase cascade will initiate a series of cellular events (e.g., mitochondrial dysfunction, DNA degradation) that result in apoptosis of a target cell. Similarly, loss of enzymes involved in glycolysis or fatty acid synthesis will result in apoptotic cell death. Activators and inhibitors of the many enzymes, proteins, and metabolic regulators such as protooncocgenes and oncogenes, p53 pathway, EGF pathway, AKT pathway, cyclin-dependent kinases that could be used to modulate intracellular signal and metabolism pathways are know to those with skill in the art.

### Positive Charge Potential of Pathway Modulators

Ideally, compounds that through large-pore cation channel receptors are positively-charged. The positive charge serves to render them impermeable to cells not displaying activated large-pore cation channel receptors as well as to facilitate the passage into cells with activated channels. In certain cases, compounds to be used to inhibit proliferation or to modulate intracellular signal transduction and metabolism pathways may require chemical modification to render a net positive charge on the molecule. The selective alkylation, guanylation or protonation of compounds is used to incorporate a proper and ideal charge into a compound to facilitate transport through large-pore cation channels. Further methods are described below.

### Compound Modification to Confer a Positive Charge

The synthesis of charge-modified compounds to allow entry through a large-pore cation channel may involve the selective protection and deprotection of alcohols, amines, ketones, sulfhydryls or carboxyl functional groups of the parent ion channel blocker, the linker, the bulky group, and/or the charged group. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a Lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994.

Charge-modified compounds can be prepared using techniques familiar to those skilled in the art. The modifications can be made, for example, by alkylation of the parent ion channel blocker using the techniques described by J. March, Advanced Organic Chemistry: Reactions, Mechanisms and Structure, John Wiley & Sons, Inc., 1992, page 617. The conversion of amino groups to guanidine groups can be accomplished using standard synthetic protocols. For example, Mosher has described a general method for preparing mono-substituted guanidines by reaction of aminoiminomethanesulfonic acid with amines (Kim et al., Tetrahedron Lett. 29:3183 (1988)). A more convenient method for guanylation of primary and secondary amines was developed by Bernatowicz employing 1H-pyrazole-1-carboxamidine hydrochloride; 1-H-pyrazole-1-(N,N′-bis(tert-butoxycarbonyl)carboxamidine; or 1-H-pyrazole-1-(N,N′-bis(benzyloxycarbonyl)carboxamidine. These reagents react with amines to give mono-substituted guanidines (see Bernatowicz et al., J. Org. Chem. 57:2497 (1992) and Bernatowicz et al., Tetrahedron Lett. 34:3389 (1993)). In addition, Thioureas and S-alkyl-isothioureas have been shown to be useful intermediates in the syntheses of substituted guanidines (Poss et al., Tetrahedron Lett. 33:5933 (1992)). In certain embodiments, the guanidine is part of a heterocyclic ring having two nitrogen atoms (see, for example, the structures below).

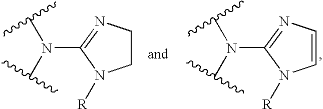

The ring system can include an alkylene or alkenylene of from 2 to 4 carbon atoms, e.g., ring systems of 5, 6, and 7-membered rings. Such ring systems can be prepared, for example, using the methods disclosed by Schlama et al., (J. Org. Chem., 62:4200 (1997)).

### Formulation of Compositions

The administration of a combination of the invention may be by any suitable means that results in the treatment of cancer. The antiproliferative agents and intracellular enzyme/pathway inhibitors and the large-pore cation channel receptor agonists may be contained in any appropriate amount in any suitable carrier substance, and are generally present in amounts totaling 1-95% by weight of the total weight of the composition. The composition may be provided in a dosage form that is suitable for oral, parenteral (e.g., intravenous, intramuscular), rectal, cutaneous, subcutaneous, topical, transdermal, sublingual, nasal, vaginal, intrathecal, epidural, or ocular administration, or by injection, inhalation, or direct contact with the nasal or oral mucosa.

Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, the first and second agents may be formulated together or separately. Desirably, the first and second agents are formulated together for the simultaneous or near simultaneous administration of the agents.

The individually or separately formulated agents can be packaged together as a kit. Non-limiting examples include but are not limited to kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (e.g., “bulk packaging”). The kit components may be assembled in cartons, blister packs, bottles, tubes, and the like.

### Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

### Topical Formulations

Compositions can also be adapted for topical use with a topical vehicle containing from between 0.0001% and 25% (w/w) or more of active ingredient(s).

In a preferred combination, the active ingredients are preferably each from between 0.0001% to 10% (w/w), more preferably from between 0.0005% to 4% (w/w) active agent. The cream can be applied one to four times daily, or as needed. For example, for prednisolone adapted for topical administration, a topical vehicle will contain from between 0.01% to 5% (w/w), preferably from between 0.01% to 2% (w/w), more preferably from between 0.01% to 1% (w/w).

Performing the methods described herein, the topical vehicle containing the combination of the invention is preferably applied to the site of cancer cell growth. For example, a cream may be applied to the skin of a subject suffering from skin cancer.

### Screening

Our discovery that certain channels expressed by and present on numerous cell types allow entry of compounds into the target cells provides a method for identifying compounds as being useful for the treatment of a wide variety of conditions. In one example, a cell is contacted with a one, two, or more compounds that activate TRPV1, TRPA1, TRPM8 and/or P2X(2/3) receptors. The same cell is also contacted with a second compound that is active when applied to the internal face of the cell (e.g., by intracellular application by liposomes) but not when applied to the external face of the cell (because of the inability of the compound to cross the cell membrane).

### EXAMPLES

The following examples are intended to illustrate the invention, and are not intended to limit it.

### Example 1

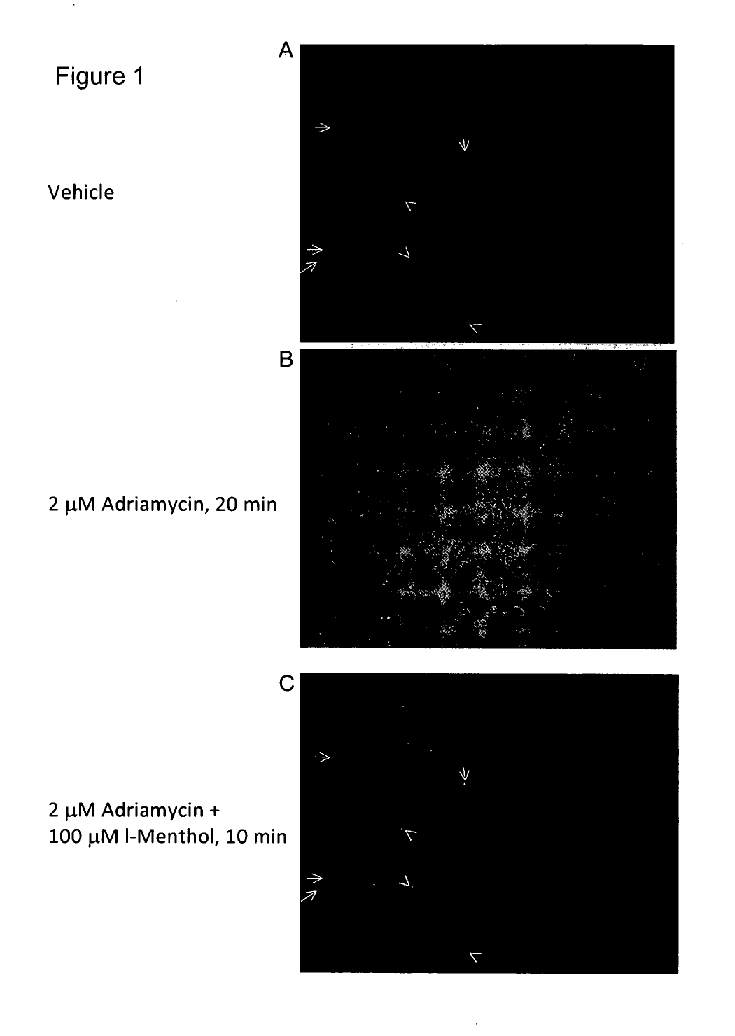

The selective administration of compounds or agents that are cytotoxic to cancer cells is accomplished by the co-administration of menthol, an agonist of the large-pore cation channel receptor TRPM8, along with the antiproliferative agent doxorubicin (Adriamycin®). As shown in FIGS. 1A-1C, co-administration of menthol with doxorubicin enabled intracellular accumulation of the doxorubicin only into those DRG neurons that express TRPM8 (FIG. 1C) when compared with cells treated with doxorubicin alone (FIG. 1B). Once doxorubicin is within a cell it enters the nucleus where it binds to DNA.

### Example 2

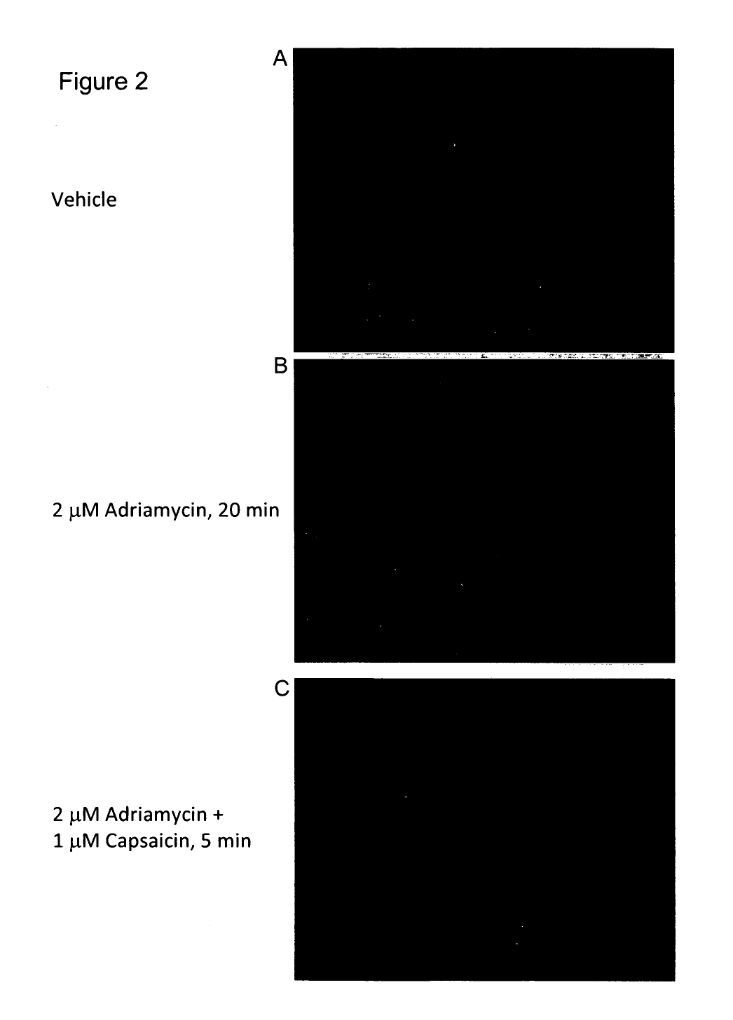

The selective administration of compounds or agents that are cytotoxic to cancer cells is accomplished by the co-administration of capsaicin, an agonist of the large-pore cation channel receptor TRPV1, along with doxorubicin (Adriamycin®). As shown in FIGS. 2A-2C, co-administration of capsaicin with doxorubicin enabled intracellular accumulation of the doxorubicin only into those DRG neurons that express TRPV1 (FIG. 2C) when compared with cells treated with doxorubicin alone (FIG. 2B).

### Example 3

The selective administration of compounds or agents that are cytotoxic to cancer cells can be accomplished by the co-administration of capsaicin, an agonist of the large-pore cation channel receptor TRPV1, along with a small, positively-charged antimitotic agent such as procainamide and related triethylamine-substituted 4-aminobenzamides, such as metoclopramide and declopramide, which induce DNA demethylation, nuclear factor-KB inhibition, and apoptosis (see, e.g., Morissette et al., “N-Substituted 4-Aminobenzamides (Procainamide Analogs): An Assessment of Multiple Cellular Effects Concerning Ion Trapping,” Mol. Pharmacol. 68:1576-1589 (2005)). Capsaicin activates the TRPV1 channel receptor and allows the antimitotic agent (e.g., procainamide), inactive while in the extracellular space, to enter the target cancer cell. Upon entry in the cell, the antimitotic agent exerts pro-apoptotic biological activity, including mitotic arrest that eventual result in the death of the cancer cell.

### Other Embodiments

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method for treating cancer in a patient, said method comprising administering to said patient:
(i) a first compound that activates a channel-forming receptor that is present on a target cell; and
(ii) an antiproliferative agent, wherein said agent is capable of entering said target cell through said receptor when said receptor is activated.

2. The methods of claims 1, wherein said cancer is esophageal cancer, prostate cancer, colon cancer, lung cancer, breast cancer, ovarian cancer, rectal cancer, bladder cancer, renal cancer, melanoma, pancreatic cancer, thyroid cancer, brain cancer, sarcomas, non-Hodgkin's lymphoma, leukemia, or endometrial cancer.

3. The method of claim 1, wherein said first channel-forming receptor is selected from the group consisting of TRPV1, TRPV6, TRPM1, TRPC1, TRPC6, TRPM4, TRPM5, TRPP8, TRPA1, P2X(2/3), and TRPM8.

4. The method of claim 3, wherein said first compound is an activator of TRPV1 receptors, said activator selected from the group consisting of capsaicin, lidocaine, eugenol, arvanil (N-arachidonoylvanillamine), anandamide, 2-aminoethoxydiphenyl borate (2APB), AM404, resiniferatoxin, phorbol 12-phenylacetate 13-acetate 20-homovanillate (PPAHV), olvanil (NE 19550), OLDA (N-oleoyldopamine), N-arachidonyldopamine (NADA), 6′-iodoresiniferatoxin (6′-IRTX), C18 N-acylethanolamines, lipoxygenase derivatives such as 12-hydroperoxyeicosatetraenoic acid, inhibitor cysteine knot (ICK) peptides (vanillotoxins), piperine, MSK195 (N-[2-(3,4-dimethylbenzyl)-3-(pivaloyloxy)propyl]-2-[4-(2-aminoethoxy)-3-methoxyphenyl]acetamide), JYL79 (N-[2-(3,4-dimethylbenzyl)-3-(pivaloyloxy)propyl]-N′-(4-hydroxy-3-methoxybenzyl)thiourea), and SU200 (N-(4-tert-butylbenzyl)-N′-(4-hydroxy-3-methoxybenzyl)thiourea), transacin, ALGRX 4975, NGX-1998, and TQ-1018, or wherein said first compound is an activator of TRPA1 receptors, said activator selected from the group consisting of cinnamaldehyde, allyl-isothiocynanate, diallyl disulfide, icilin, cinnamon oil, wintergreen oil, clove oil, acrolein, and mustard oil, or wherein said first compound is an activator of P2X receptors, said activator selected from the group consisting of ATP, 2-methylthio-ATP, T and 3′-O-(4-benzoylbenzoyl)-ATP, and ATP5′-O-(3-thiotriphosphate).

5-8. (canceled)

9. The method of claim 1, wherein said antiproliferative agent is selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, histone acetyltransferase inhibitors, metalloproteinase inhibitors, ribonucleoside reductase inhibitors, photodynamic agents, and tyrosine kinase inhibitors.

10-24. (canceled)

25. A method for modulating intracellular signal transduction and metabolic pathways in a patient, said method comprising administering to said patient:
(i) a first compound that activates a channel-forming receptor that is present on a target cell; and
(ii) a second compound that inhibits or activates an intracellular signal transduction or metabolic pathway, wherein said second compound is capable of entering the target cell through said receptor when said receptor is activated and does not substantially inhibit said pathway when applied extracellularly in the absence of said first compound.

26. The method of claim 25, wherein said channel-forming receptor is selected from the group consisting of TRPV1, TRPV6, TRPM1, TRPC1, TRPC6, TRPM4, TRPM5, TRPP8, TRPA1, P2X(2/3), and TRPM8.

27. The method of claim 26, wherein said first compound is an activator of TRPV1 receptors, said activator selected from the group consisting of capsaicin, lidocaine, eugenol, arvanil (N-arachidonoylvanillamine), anandamide, 2-aminoethoxydiphenyl borate (2APB), AM404, resiniferatoxin, phorbol 12-phenylacetate 13-acetate 20-homovanillate (PPAHV), olvanil (NE 19550), OLDA (N-oleoyldopamine), N-arachidonyldopamine (NADA), 6′-iodoresiniferatoxin (6′-IRTX), C18 N-acylethanolamines, lipoxygenase derivatives such as 12-hydroperoxyeicosatetraenoic acid, inhibitor cysteine knot (ICK) peptides (vanillotoxins), piperine, MSK195 (N-[2-(3,4-dimethylbenzyl)-3-(pivaloyloxy)propyl]-2-[4-(2-aminoethoxy)-3-methoxyphenyl]acetamide), JYL79 (N-[2-(3,4-dimethylbenzyl)-3-(pivaloyloxy)propyl]-N′-(4-hydroxy-3-methoxybenzyl)thiourea), and SU200 (N-(4-tert-butylbenzyl)-N′-(4-hydroxy-3-methoxybenzyl)thiourea), transacin, ALGRX 4975, NGX-1998, and TQ-1018, or wherein said first compound is an activator of TRPA1 receptors, said activator selected from the group consisting of cinnamaldehyde, allyl-isothiocynanate, diallyl disulfide, icilin, cinnamon oil, wintergreen oil, clove oil, acrolein, and mustard oil, or wherein said first compound is an activator of P2X receptors, said activator selected from the group consisting of ATP, 2-methylthio-ATP, 2′ and 3′-O-(4-benzoylbenzoyl)-ATP, and ATP5′-O-(3-thiotriphosphate).

28-31. (canceled)

32. The method of claim 25, wherein said second compound is an enzyme inhibitor or activator, an inhibitor or activator of an intracellular protein kinase, or an inhibitor or activator of an intracellular protein phosphatase.

33-40. (canceled)

41. A composition for treating cancer in a patient comprising:
(i) a first compound that activates a channel-forming receptor that is present on a target cell; and
(ii) a second compound that is an antiproliferative agent and/or inhibits or activates an intracellular signal transduction or metabolic pathway, wherein said second compound is capable of entering the target cell through said receptor when said receptor is activated and does not substantially inhibit said pathway when applied extracellularly in the absence of said first compound.

42. The composition of claim 41, wherein said channel-forming receptor is selected from the group consisting of TRPV1, TRPV6, TRPM1, TRPC1, TRPC6, TRPM4, TRPM5, TRPP8, TRPA1, P2X(2/3), and TRPM8.

43. The compositions of claim 42, wherein said first compound is an activator of TRPV1 receptors, said activator selected from the group consisting of capsaicin, lidocaine, eugenol, arvanil (N-arachidonoylvanillamine), anandamide, 2-aminoethoxydiphenyl borate (2APB), AM404, resiniferatoxin, phorbol 12-phenylacetate 13-acetate 20-homovanillate (PPAHV), olvanil (NE 19550), OLDA (N-oleoyldopamine), N-arachidonyldopamine (NADA), 6′-iodoresiniferatoxin (6′-IRTX), C18 N-acylethanolamines, lipoxygenase derivatives such as 12-hydroperoxyeicosatetraenoic acid, inhibitor cysteine knot (ICK) peptides (vanillotoxins), piperine, MSK195 (N-[2-(3,4-dimethylbenzyl)-3-(pivaloyloxy)propyl]-2-[4-(2-aminoethoxy)-3-methoxyphenyl]acetamide), JYL79 (N-[2-(3,4-dimethylbenzyl)-3-(pivaloyloxy)propyl]-N′-(4-hydroxy-3-methoxybenzyl)thiourea), and SU200 (N-(4-tert-butylbenzyl)-N′-(4-hydroxy-3-methoxybenzyl)thiourea), transacin, ALGRX 4975, NGX-1998, and TQ-1018, or said first compound is an activator of TRPA1 receptors, said activator selected from the group consisting of cinnamaldehyde, allyl-isothiocynanate, diallyl disulfide, icilin, cinnamon oil, wintergreen oil, clove oil, acrolein, and mustard oil, or said first compound is an activator of P2X receptors, said activator selected from the group consisting of ATP, 2-methylthio-ATP, 2′ and 3′-O-(4-benzoylbenzoyl)-ATP, and ATP5′-O-(3-thiotriphosphate).

44-47. (canceled)

48. The composition of claim 41, wherein said antiproliferative agent is selected from the group consisting of alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, histone acetyltransferase inhibitors, metalloproteinase inhibitors, ribonucleoside reductase inhibitors, photodynamic agents, and tyrosine kinase inhibitors.

49-69. (canceled)

70. The composition of claim 41, wherein said second compound is an enzyme inhibitor or activator, an inhibitor or activator of an intracellular protein kinase, or an inhibitor or activator of an intracellular protein phosphatase.

71-81. (canceled)
